(19) United States
(12) Patent Application Publication
(10) Pub. No.: US 2006/0102178 A1
(43) Pub. Date: May 18, 2006

(54) Device for recording the parameters of an aerosol in particular in inhalation therapy devices

(75) Inventors: Franz Feiner, Munchen (DE); Markus Borgschulte, Munchen (DE)

Correspondence Address: WOLF GREENFIELD & SACKS, PC;FEDERAL RESERVE PLAZA, 600 ATLANTIC AVENUE, BOSTON, MA, 02210-2206, US

(73) Assignee: PARI GmbH Spezialisten fur effektive Inhalation, Starnberg (DE)

(21) Appl. No.: 10/517,759

(22) PCT Filed: Jun. 6, 2003
(86) PCT No.: PCT/EP03/05998

§ 371 Date: Nov. 4, 2005

(30) Foreign Application Priority Data
Jun. 13, 2002 (DE) 102 26 334.5

(51) Int. Cl. A61M 11/00; A61M 15/00

(52) U.S. Cl. 128/203.12; 128/200.14

(57) ABSTRACT

The invention relates to a device for detecting the parameters of an aerosol, in particular in inhalation therapy devices, whereby light is shone through a translucent material but not through a transparent material in a detection region and is detected and analysed by means of two receivers.

The invention relates to a device for recording the parameters of an aerosol, in particular in inhalation therapy devices.

Known from DE 100 22 795 A is a breath-controlled inhalation therapy device, in which an infrared light transmitter is disposed adjacent to an infrared light receiver in an opening in the mouthpiece of the therapy device such that the infrared light emitted by the transmitter arrives in a detection area in which a still aerosol is located. The infrared light is reflected by the aerosol particles or droplets and arrives at the receiver which emits an output signal that corresponds to the density of the aerosol. The transparent surface through which the infrared light is emitted and the reflected infrared light is received is disposed in the interior of the therapy device, i.e., for example, in the internal space of the mouthpiece, so that in the known inhalation therapy device, the light shining in can directly arrive at the aerosol droplets from the transmitter and the light reflected by these droplets can arrive at the receiver. The transmitter and receiver work with optical-imaging radiation.

Although the known device is basically suitable for ensuring breath-controlled nebulisation, and although the control method described in DE 100 22 795 A can be reliably realised, it has shown that owing to the adhesion of larger and smaller droplets to the transparent surface through which the light passes, the analysis of the output signal of the receiver during the control process is comparatively complicated, in particular if the precision desired for therapeutic applications is supposed to be achieved.

There is therefore demand for an inhalation therapy device in which detection of aerosol particles or droplets in a given spatial area in the therapy device occurs in a manner that allows an analysis, in particular control of nebulisation, to be designed more simply and thus more economically.

In view of the above, it is the object of the invention to specify a device for recording the parameters of an aerosol and, in particular, an inhalation therapy device having such a device, in which the analysis of detection signals and the control of nebulisation based thereon is simplified.

This aim is achieved by means of a device having the features described in patent claim 1. Advantageous embodiments can be seen in the sub-claims.

It is an important factor of the invention that shining in, for example of light into the detection area, occurs through a translucent material and not through a transparent material. The beam expansion linked therewith leads to a surprisingly high insensitivity to aerosol particles or droplets that adhere to the material through which shining occurs, without the analysability of the measurement signals being affected. This considerable insensitivity to impaction is based on an averaging over large spatial areas that is linked with the beam expansion. According to the invention, the transmitter and receiver work with non-imaging radiation.

If shining in occurs in a clocked manner or intermittently, the effect of ambient light can be determined using a reference measurement in the dark phases and can be later consulted when analysing the measurement signals in the light phases. The reaction speed of detection is thereby determined by means of the clock frequency.

Since at least two receivers are provided according to the invention, the output signals of the receivers could be mathematically linked in the analysis, for example by forming a quotient, whereby reducing the effect of ambient light and/or temperature fluctuations. It is advantageous in this regard for one receiver to be disposed in the main beam direction of the transmitter and for the other to be disposed substantially perpendicularly to the main beam direction.

Reduction of the effect of ambient light can also occur by means of series-connected long-pass filters, preferably on the receivers.

In the following description of embodiments, the invention is explained in more detail by means of the figures.

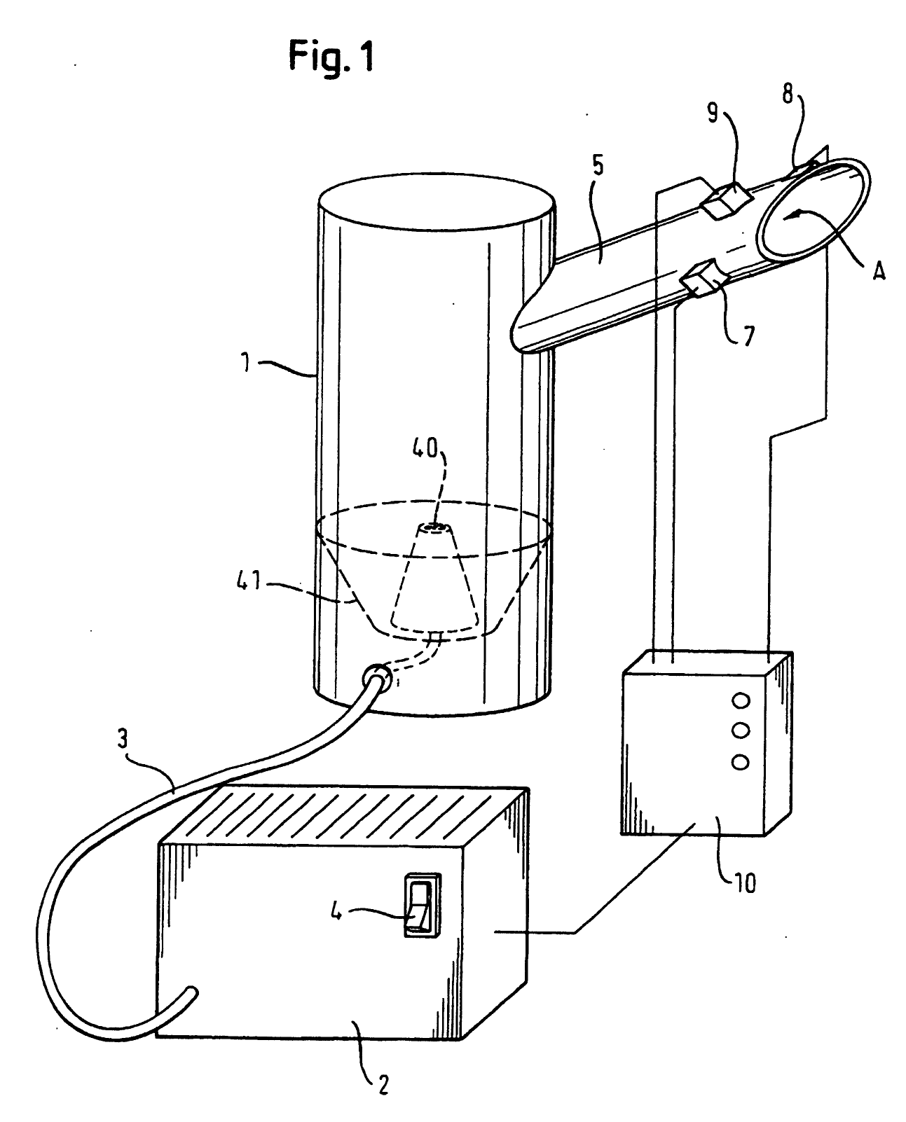

FIG. 1 shows an inhalation therapy device having a recording device according to the invention,

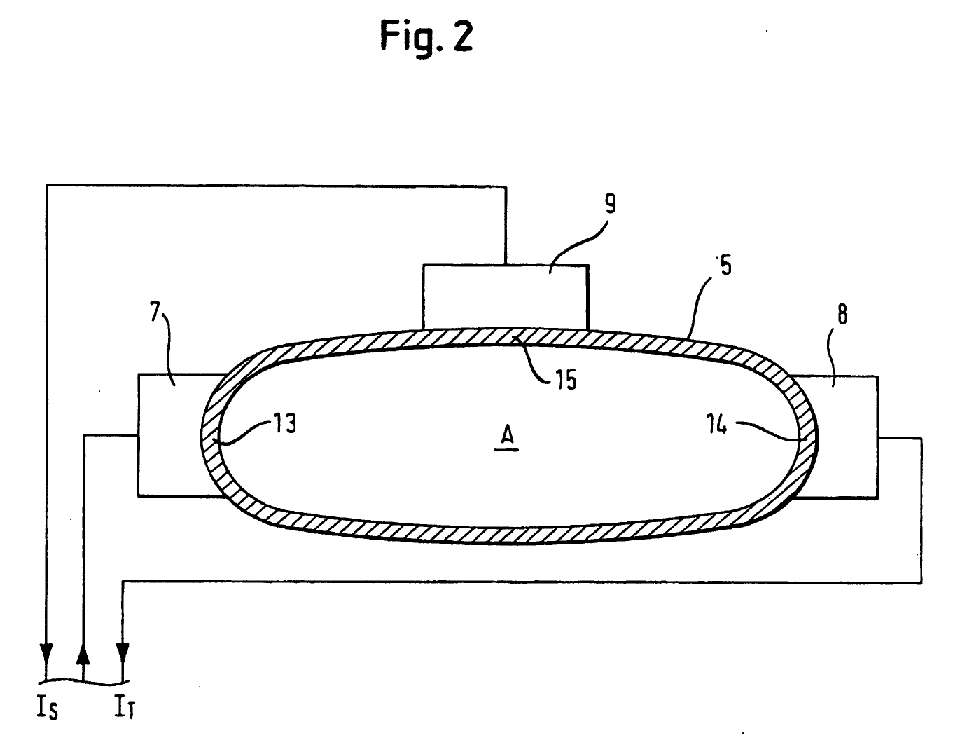

FIG. 2 shows a view of the arrangement of the transmitter and receivers of a recording device according to the invention, and

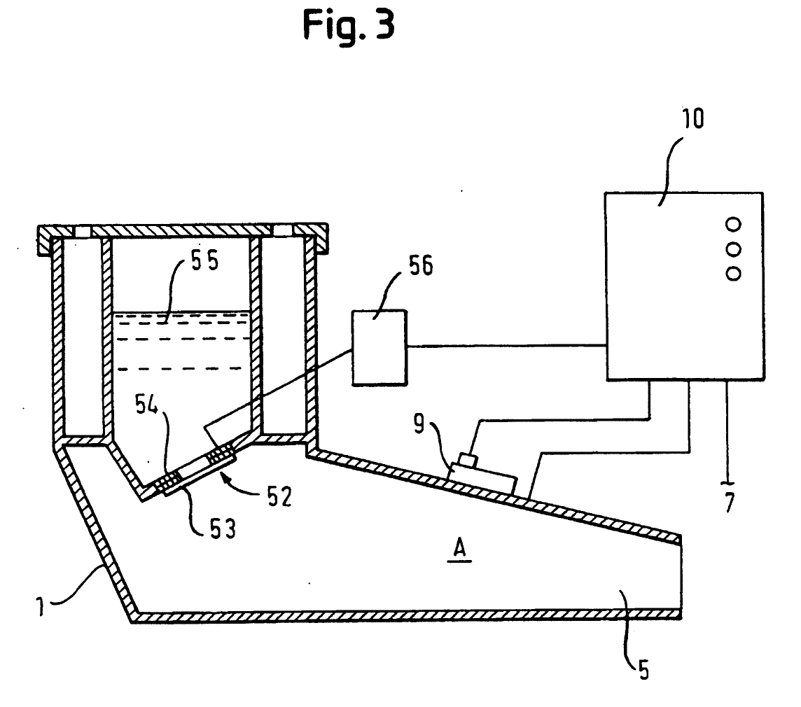

FIG. 3 shows a further inhalation therapy device having a recording device according to the invention.

A general view of an embodiment of an inhalation therapy device according to the invention is shown in FIG. 1. In this embodiment, the inhalation therapy device comprises a nebuliser 1 that accommodates a nebuliser nozzle 40 in its interior, which acts as the primary aerosol generator. The nebuliser nozzle 40 is supplied with compressed air by a compressor 2 via a tube 3 when the compressor is switched on. The nebuliser nozzle then sucks in the liquid to be nebulised from a storage container 41 in which it is disposed. The compressor can be manually switched on via a rocker switch 4. A mouthpiece 5 is attached to the nebuliser 1, via which a patient inhales the aerosol generated in the nebuliser by the nebuliser nozzle.

Alternatively, an inhalation therapy device having a membrane nebuliser 52 can also be used instead of the inhalation therapy device with a nebuliser nozzle; such an inhalation therapy device is exemplified in FIG. 3. In order to generate the aerosol, the inhalation therapy device 1 according to FIG. 3 comprises a membrane nebuliser 52 having a membrane 53 attached in a ring shape to a piezo element 54. The liquid 55 to be nebulised is contiguous with a membrane 53 and is nebulised through the openings of said membrane 53 when the piezo element 54 causes the membrane to oscillate. The piezo element 54 is activated for this purpose by means of an excitation device 56. The inhalation therapy device 1 shown in FIG. 2 also comprises a mouthpiece 5, via which the patient inhales the aerosol generated by the membrane nebuliser.

As shown in FIG. 1, a transmitting means 7, a first receiving means 8 and a second receiving means 9 are disposed, according to the invention, on the mouthpiece 5. Owing to the spatial arrangement of the transmitting means and the two receiving means, an area is defined in the interior of the mouthpiece, in which the parameters of an aerosol that rests here can be recorded by the transmitter/receiver arrangement. This area is referred to as aerosol resting area A in this description of an embodiment of the invention.

The transmitting means 7 emits light, preferably infrared light (or another suitable radiation), into the interior of the mouthpiece 5, namely into the aerosol resting area. The first receiving means 8 receives the proportion of light that penetrates the mouthpiece 5 essentially unscattered and releases a first output signal I_{T} which is supplied to an analysis/control unit 10. The first receiving means 8 is arranged, for example, in the main beam direction of the transmitting means 7. The second receiving means 9 receives the proportion of light that is scattered by aerosol particles or droplets and releases a second output signal I_{S} which is also supplied to the analysis/control means 10. The second receiving means 9 is arranged, for example, at an angle, preferably perpendicularly to the main beam direction of the transmitting means 7.

A cross-section through the mouthpiece 5 of the embodiment of an inhalation therapy device according to the invention as seen in FIG. 1 is shown in FIG. 2.

It can be seen in FIG. 2 how the transmitting means 7 is arranged on the wall of the mouthpiece 5 such that the transmitting means 7 emits light into the interior of the mouthpiece 5 through the first translucent wall section 13. In this case, which is particularly economical, the exit surface on the transmitter for the emitted radiation can be as desired. If the mouthpiece is made of a transparent material, the transmitting means 7 is provided with a translucent surface, through which the radiation of the transmitting means 7 passes and which assumes the function of the first translucent wall section 13. If the mouthpiece is made of a non-transparent material, the translucent surface of the transmitting means 7 is arranged in an opening in the wall of the mouthpiece, which is preferably completely closed by the transmitting means. According to the invention, the aerosol resting area, labelled A in FIG. 2, thus achieves non-imaging radiation since the light of the transmitter is guided through a translucent material, for example an opaline plastic.

It can furthermore be seen in FIG. 2, how the first receiving means 8 is arranged on the wall of the mouthpiece 5 such that said first receiving means 8 primarily receives, through a second wall section 14 of the mouthpiece 5, the proportion of the light emitted into the aerosol resting area A, which comes from the transmitting means 7 and passes through the aerosol resting area A, i.e. in this case the interior of the mouthpiece 5, in an unscattered manner. This light is called transmission light TL in this description. If no aerosol is present in the interior of the mouthpiece 5, the light emitted by the transmitting means 7 reaches the first receiving means 8 practically uninterrupted, said receiving means 8 thereupon emitting a high output signal I_{TL}. As the aerosol density in the aerosol resting area A increases, the light emitted by the transmitting means 7 will be scattered to a greater extent such that less light reaches the first receiving means 8. The output signal I_{TL} of the first receiving means 8 decreases as the aerosol density in the aerosol resting area increases.

It is less important for the arrangement of the first receiving means 8 on the mouthpiece 5 whether the mouthpiece is produced from a transparent or translucent material. However, the light preferably falls through a translucent material into the first receiving means 8. Reference is made in this regard to the explanations regarding the transmitting means 7 and the first wall section 13, which accordingly also apply to the first receiving means 8 and the second wall section 14. Particularly economical is again a mouthpiece 5 that is made of a translucent material, which renders a further translucent material on the first receiving means 8 unnecessary.

Finally, it can be seen in FIG. 2 how the second receiving means 9 is arranged on a third wall section 15 of the mouthpiece 5 such that said second receiving means 9 primarily receives, through the second wall section 15, the proportion of light emitted into the aerosol resting area A, which comes from the transmitting means 7 and is scattered by the aerosol particles or droplets. This light is called scattered light SL in this description. If no aerosol is present in the aerosol resting area A, i.e. in the interior of the mouthpiece 5 in the embodiment described here, only a small amount of the light emitted by the transmitting means 7 reaches the second receiving means 9; the second receiving means 9 thereupon emits a low output signal I_{SL}. Since the light emitted by the transmitting means 7 is scattered to a greater extent as the aerosol density in the aerosol resting area A increases, increasingly more light reaches the second receiving means 9. The output signal I_{SL} of the second receiving means 9 increases as the aerosol density in the aerosol resting area increases.

It is less important for the arrangement of the second receiving means 9 on the mouthpiece 5 whether the mouthpiece is produced from a transparent or translucent material. However, the light preferably falls through a translucent material into the second receiving means 9. Reference is made in this regard to the explanations regarding the transmitting means 7 and the first wall section 13, which accordingly also apply to the second receiving means 9 and the second wall section 15. Particularly economical is again a mouthpiece 5 that is made of a translucent material, which renders a further translucent material on the second receiving means 9 unnecessary.

The first and second output signals (I_{TL}, I_{SL}) of the receiving means 8 and 9 are supplied to the control means 10, which analyses the first and second output signals (I_{TL}, I_{SL}) to determine the parameters of an aerosol in the aerosol resting area A.

A first analysis can occur to the effect that the control means 10 determines whether or not an aerosol is present in the aerosol resting area A. A high first output signal I_{TL} and a low second output signal I_{SL} indicate that almost no aerosol is present in the aerosol resting area A. A low first output signal I_{TL} and a high second output signal I_{SL} indicate that an aerosol is present in the aerosol resting area A. Therefore, the presence of the aerosol in the aerosol resting area A can be determined as a first parameter of said aerosol.

If the second output signal I_{SL} increases and the first output signal I_{TL} decreases, this indicates that an aerosol is present in the aerosol resting area A whose density is increasing. If the first output signal I_{TL} increases and the second output signal I_{SL} decreases, this indicates that an aerosol is present in the aerosol resting area A whose density is decreasing. Therefore, the change in the density of the aerosol in the aerosol resting area A can be determined as a second parameter of said aerosol.

If calibration is carried out, the aerosol density is also to be absolutely determined as a third parameter from the output signals I_{SL} and I_{SL}.

The control method described in DE 100 22 795 A can essentially also be carried out based on the two output signals I_{SL} and I_{SL}. Particularly suitable in this regard is an inhalation therapy device having a membrane nebuliser, as shown, for example, in FIG. 3. In order to control nebulisation, the control means 10 is connected with the compressor 2 and with the excitation device 56.

For this purpose, the quotient
Q_{A}=I_{SL}/I_{TL}
is preferably formed from the first and second output signals I_{SL} and I_{SL} in the control means 10. The effect of ambient light and temperature fluctuations on the transmitting and receiving means 7, 8 and 9 is thereby eliminated, or is at least clearly reduced.

For example, the presence of aerosol in the aerosol resting area A defined by the transmitting and receiving means can be determined in that the control means 10 determines whether the quotient is above a threshold Q_{Amin}, which is only exceeded if a sufficient amount of aerosol particles or droplets are present in the mouthpiece 5 between the transmitter 7 and the receivers 8, 9.

To further improve insensitivity to ambient light, the transmitting means 7 is intermittently operated by the control means 10 such that first time periods Z1, in which the transmitting means 7 emits light into the aerosol resting area A, alternate with second time periods Z2, in which the transmitter means 7 does not emit any light. The output signals of the first and second receiving means 8 and 9 are different in both time periods.

In one of the second time periods Z2, in which no light is emitted into the aerosol resting area A by the transmitting means 7, only ambient light reaches the first and second receiving means 8 and 9, which shines, for example, through the translucent material of the mouthpiece 5 or the mouthpiece opening into the aerosol resting area A and arrives at the first or second receiving means 8 and 9. In one of the first time periods Z1, in which the transmitting means 7 emits light into the aerosol resting area A, transmission light TL and scattered light SL in addition to ambient light also reach the first and second receiving means 8 and 9. The output signal of the first and second receiving means 8 and 9 thus changes at least as regards how high it is. The output signals I_{TL} and I_{SL} in the time periods Z1 and Z2 can be detected by the control means 10 and can be allocated to time periods Z1 and Z2 since the control means 10 determines the sequence of the time periods via activation of the transmitting means 7.

When the transmitting means 7 is operated intermittently, it is possible to determine, in the second time periods Z2 in which the transmitting means 7 does not emit any light into the aerosol resting area A, the proportion of ambient light contained in the output signal of the first and second receiving means 8 and 9. The output signals I_{TLU} and I_{SLU} occurring in the second time periods Z2 are attributed to the ambient light that reaches the receivers. The control means 10 takes into account the proportions of the output signals I_{TLU} and I_{SLU} attributed to the ambient light in the first time periods Z1, in which the transmitting means 7 emits light into the aerosol resting area A, in order to eliminate the proportion of ambient light in the output signals I_{TL} and I_{SL}, for example in a manner in which the differences (I_{TL}−I_{TLU}) and (I_{SL}−I_{SLU}) are formed. The effect of ambient light is further reduced in this manner. In this case,

$${Q}_{A} = \frac{( {I}_{SL} - {I}_{SLU} )}{( {I}_{TL} - {I}_{TLU} )}$$

is formed as the quotient. It is thereby ensured, owing to the repeated succession of the first and second time periods Z1 and Z2, that fluctuations in ambient light are also taken into consideration.

The intermittent operation of the transmitting means 7 furthermore makes it possible to check the operability of the transmitting means 7 and the receiving means 8 and 9 since alternating the operating state of the transmitting means 7 must lead to a change in the output signal of the receiving means. If there is no change, a defect in the transmitter or in one of the receivers can be concluded.

1. A device for detecting the parameters of an aerosol, in particular in an inhalation therapy device, comprising
a. a transmitting means
i. which is disposed on a body that at least partially surrounds an aerosol resting area, and
ii. which emits radiation into said aerosol resting area through a translucent material;
b. a first receiving means,
i. which is disposed on the body (that at least partially surrounds said aerosol resting area,
ii. which is disposed in relation to said transmitting means so as to primarily receive transmission radiation, and
iii. which emits a first analysis signal that corresponds to the intensity of the received transmission radiation;
c. a second receiving means,
i. which is disposed on the body that at least partially surrounds said aerosol resting area,
ii. which is disposed in relation to said transmitting means so as to primarily receive scattered radiation, and
iii. which emits a second analysis signal that corresponds to the intensity of the received scattered radiation; and
d. a control means, to which the first and second output signals are supplied and which analyses the first and second output signals in order to determine the parameters of an aerosol in said aerosol resting area.

2. A device for detecting the parameters of an aerosol according to claim 1, wherein the transmitting means emits the radiation through a first translucent wall section of the body surrounding the aerosol resting area.

3. A device for detecting the parameters of an aerosol according to claim 1, wherein the first receiving means receives the transmission radiation through a second wall section of the body surrounding the aerosol resting area.

4. A device for detecting the parameters of an aerosol according to claim 1, wherein the second receiving means receives the scattered radiation through a third wall section of the body surrounding the aerosol resting area.

5. A device for detecting the parameters of an aerosol according to claim 1, wherein the body surrounding the aerosol resting area is made of a translucent material.

6. A device for detecting the parameters of an aerosol according to claim 1, wherein the body surrounding the aerosol resting area is made of a transparent material and the transmitting means is provided with a surface made of a translucent material, through which radiation is emitted.

7. A device for detecting the parameters of an aerosol according to claim 1, wherein the first receiving means is provided with a surface made of a translucent material, through which the radiation is received.

8. A device for detecting the parameters of an aerosol according to claim 1, wherein the second receiving means is provided with a surface made of a translucent material, through which the radiation is received.

9. A device for detecting the parameters of an aerosol according to claim 1, wherein the control means activates the transmitting means to emit the radiation into the aerosol resting area.

10. A device for detecting the parameters of an aerosol according to claim 9, wherein the control means activates the transmitting means such that first time periods, in which the transmitting means emits radiation into the aerosol resting area, alternate with second time periods, in which the transmitting means does not emit radiation into the aerosol resting area.

11. A device for detecting the parameters of an aerosol according to claim 9, wherein in the second time periods, the control means determines the proportion of ambient light in the output signals of the first and/or second receiving means.

12. A device for detecting the parameters of an aerosol according to claim 11, wherein the control means makes use of the proportion of ambient light when analysing the output signals of the first and second receiving means.

13. A device for detecting the parameters of an aerosol according to claim 12, wherein the control means forms the difference of the output signal of the first receiving means and the first ambient light proportion and/or the difference of the output signal of the second receiving means and the second ambient light proportion.

14. A device for detecting the parameters of an aerosol according to claim 13, wherein the control means forms the quotient from the difference of the output signal of the second receiving means and the second ambient light proportion and the difference of the output signal of the first receiving means and the first ambient light proportion.

15. A device for detecting the parameters of an aerosol according to claim 1, wherein the control means forms the quotient from the output signal of the second receiving means and the output signal of the first receiving means.

16. A device for detecting the parameters of an aerosol, according to claim 1, wherein the radiation emitted by the transmitting means is light, in particular infrared light.

17. Inhalation therapy device having a device for detecting the parameters of an aerosol according to claim 1, wherein the body surrounding the aerosol resting area is a mouthpiece of the inhalation therapy device.

18. Inhalation therapy device according to claim 17, wherein a nebuliser nozzle or a membrane nebuliser is provided.

19. Inhalation therapy device according to claim 18, wherein the control means is connected with a compressor for the nebuliser nozzle or with an excitation device for the membrane nebuliser.
